(19) United States
(12) Patent Application Publication
(10) Pub. No.: US 2013/0236365 A1
(43) Pub. Date: Sep. 12, 2013

(54) ADSORPTIVE PHOTO-CATALYTIC OXIDATION AIR PURIFICATION DEVICE

(71) Applicant: Christopher C. Willette, Tequesta, FL (US)

(72) Inventor: Christopher C. Willette, Tequesta, FL (US)

(21) Appl. No.: 13/870,752

(22) Filed: Apr. 25, 2013

Related U.S. Application Data
(63) Continuation of application No. 12/793,328, filed on Jun. 3, 2010, now abandoned.
(60) Provisional application No. 61/183,614, filed on Jun. 3, 2009.

(51) Int. Cl. A61L 9/20

(52) U.S. Cl. CPC A61L 9/205
USPC 422/121

(57) ABSTRACT

An air purification system formed from an adsorptive photo-catalytic oxidation device and a method of regenerating the oxidation device is disclosed. The air purification may be configured to be installed within an air duct of a central air handling system. The air purification system may also include an ultraviolet light emitted by the ultraviolet light source to breakdown captured volatile organic compounds into elemental carbon dioxide and water vapor and to irradiate air moving past the ultraviolet light and surfaces to reduce contaminants The ultraviolet light source may be positioned to expose the adsorptive photo-catalytic oxidation device to ultraviolet light emitted by the ultraviolet light source to breakdown captured volatile organic compounds into elemental carbon dioxide and water vapor and to irradiate air moving past the ultraviolet light to reduce contaminants.

### CROSS REFERENCE TO RELATED APPLICATION

This application is a continuation of U.S. patent application Ser. No. 12/793,328, filed Jun. 3, 2010 and claims the benefit of U.S. Provisional Patent Application Ser. No. 61/183,614, filed Jun. 3, 2009, the entireties of which is incorporated by reference herein.

### FIELD OF THE INVENTION

This disclosure is directed generally to air purification systems, and more particularly, to air purification systems for removal of volatile organic compounds.

### BACKGROUND

Recent studies have shown that the level of invisible airborne organic chemical and odor contaminates in our indoor air is generally two to five times higher than the levels found outdoors. These potentially harmful contaminates, known as volatile organic compounds are a large group of carbon-based chemicals that easily evaporate at room temperature. While most people can smell high levels of some volatile organic compounds, other volatile organic compounds have no odor. Odor does not indicate the level of risk from inhalation of this group of chemicals. There are thousands of different volatile organic compounds produced and used in our daily lives. Some common examples include: acetone, benzene, ethylene glycol, formaldehyde, methylene chloride, perchloroethylene, toluene and xylene. Volatile organic compounds are often released from products such as building materials, carpets, adhesives, upholstery fabrics, vinyl floors, composite wood products, paints, varnishes, sealing caulks, glues, carpet cleaning solvent, home care products, air fresheners, air cleaners that produce ozone, cleaning and disinfecting chemicals, cosmetics, smoking, fireplaces, fuel oil, gasoline, moth balls and vehicle exhaust from running a car in an attached garage. Daily activities that release volatile organic compounds include: cooking, dry clean clothes, carpet cleaning, household cleaning, hobbies, crafts, newspapers, magazines, non-electric space heaters, photocopiers, smoking, stored paints and chemicals, and wood burning stoves

The health risks from inhaling any chemical depend on how much is in the air, how long and how often a person inhales the chemical. Scientists look at short-term (acute) exposures as an exposure between a period of hours to a period of days or long-term (chronic) exposures as years to even a lifetime. Breathing low levels of volatile organic compounds for long periods of time may increase the risk of health problems for some people. Several studies suggest that exposure to volatile organic compounds may make symptoms worse in people who have asthma or are particularly sensitive to chemicals. Short-term exposure (acute) to high levels of volatile organic compounds may cause eye, nose and throat irritation, headaches, nausea, vomiting, dizziness or worsening of asthma symptoms. Long-term exposure (chronic) to high levels of volatile organic compounds creates an increased risk of cancer, liver damage, kidney damage, and central nervous system damage. Thus, a need exists for removing volatile organic compounds from our air supplies.

### SUMMARY OF THE INVENTION

An air purification system formed from an adsorptive photo-catalytic oxidation device and a method of regenerating the oxidation device is disclosed. The air purification system may be configured to be installed within an air duct of a central air handling system. The air purification system may also include an ultraviolet light emitted by an ultraviolet light source to breakdown captured volatile organic compounds into elemental carbon dioxide and water vapor and to irradiate air moving past the ultraviolet light and surfaces to reduce contaminants The ultraviolet light source may be positioned to expose the adsorptive photo-catalytic oxidation device to ultraviolet light emitted by the ultraviolet light source. The air purification system controls and reduces indoor related volatile organic compounds by first adsorbing the airborne contaminate into the adsorptive photo-catalytic oxidation device, which may be an activated carbon honeycomb monolithic cell, and then breaking the volatile organic compound contaminate down via a photo-catalytic oxidation process.

The air purification system may include a housing having one or more adsorptive photo-catalytic oxidation device. The housing may be formed from a generally rectangular box containing the at least one adsorptive photo-catalytic oxidation device, and the ultraviolet light source extends from the housing. A deflector may extend from the housing along the ultraviolet light source to deflect air through the adsorptive photo-catalytic oxidation device and to deflect ultraviolet radiation emitted from the ultraviolet light source. The ultraviolet light source may be positioned to expose the adsorptive photo-catalytic oxidation device to ultraviolet light emitted by the ultraviolet light source to breakdown captured volatile organic compounds into elemental carbon dioxide and water vapor and to irradiate air moving past the ultraviolet light and surfaces to reduce contaminants

The adsorptive photo-catalytic oxidation device may be formed from an adsorption media. In one embodiment, the adsorption media may be an activated carbon monolithic material. The adsorptive photo-catalytic oxidation device maybe formed from a highly absorbent form of activated carbon configured in a low pressure drop honeycomb monolith. The air purification system may also include a coating of a regenerative photo-catalyst on the adsorptive photo-catalytic oxidation device. The coating of a regenerative photo-catalyst may be, but is not limited to being, an ultraviolet reactive titanium dioxide based semi-conductor photo-catalyst.

The air purification system may be installed in a central air handling system. In particular, the air purification system may be installed in an air duct extending therefrom, the housing and ultraviolet light source may be positioned in the air duct. The adsorptive photo-catalytic oxidation device may capture volatile organic compounds in the air being passed through the air duct. The ultraviolet light may kill contaminants, including, but not limited to, algal, fungal, bacterial, and viral contamination. The ultraviolet light may also regenerate the adsorptive photo-catalytic oxidation device.

An advantage of this invention is that the air purification system may remove volatile organic compounds from air being passed through the air purification system and may remove contaminants, such as, but not limited to, algal, fungal, bacterial, and viral contamination from the air and surfaces with the use of ultraviolet light.

Another advantage of this invention is that the ultraviolet light regenerates the adsorptive photo-catalytic oxidation device.

Yet another advantage of this invention is that the air purification system may be sold as a kit to retrofit currently existing central air handling systems.

These and other components are described in more detail below.

### BRIEF DESCRIPTION OF THE DRAWINGS

The accompanying drawings, which are incorporated in and form a part of the specification, illustrate embodiments of the presently disclosed invention and, together with the description, disclose the principles of the invention.

FIG. 1 is a perspective view of an air purification system for removing volatile organic compounds and for removing contaminants from the air being moved by the air purification system.

FIG. 2 is a side perspective view with partial cutaway sections of the air purification system installed in a duct of a central air handling system immediately downstream of an air handler.

FIG. 3 is a detailed perspective view of an adsorptive photocatalytic matrix of the air purification system taken at detail 3 in FIG. 1.

FIG. 4 is a diagram of the adsorption process of the air purification system.

FIG. 5 is a diagram of the regeneration process of the air purification system.

FIG. 6 is a graph of an air purification system test on the removal of toluene from air.

FIG. 7 is a graph of an air purification system test on the removal of odors from air.

FIG. 8 is a graph of an air purification system test showing the amount of volatile organic compounds removed from air.

### DETAILED DESCRIPTION OF THE INVENTION

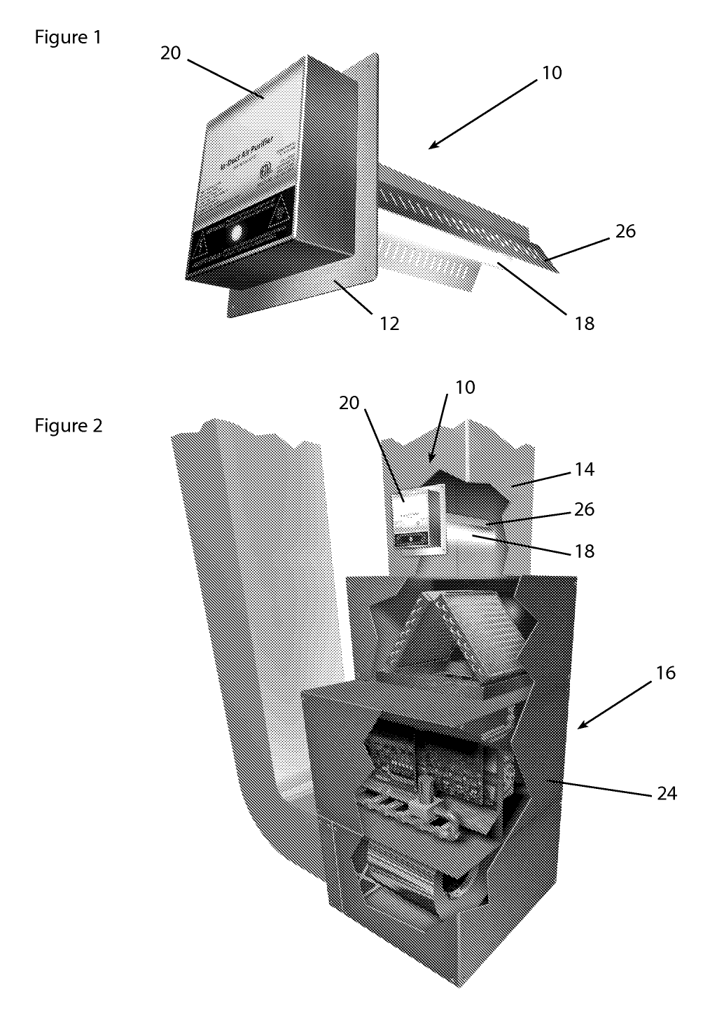

As shown in FIGS. 1-8, an air purification system 10 formed from an adsorptive photo-catalytic oxidation device 12 and a method of regenerating the oxidation device 12 is disclosed. The air purification system 10 may be configured to be installed within an air duct 14 of a central air handling system 16. The air purification system 10 may also include an ultraviolet light emitted by an ultraviolet light source 18 to breakdown captured volatile organic compounds into elemental carbon dioxide and water vapor and to irradiate air moving past the ultraviolet light 18 and local surfaces to reduce contaminants The ultraviolet light source 18 may be positioned to expose the adsorptive photo-catalytic oxidation device 12 to ultraviolet light emitted by the ultraviolet light source 18. The air purification system 10 controls and reduces indoor related volatile organic compounds by first adsorbing the airborne contaminate into the adsorptive photo-catalytic oxidation device 12, which may be an activated carbon honeycomb monolithic cell, and then breaking the volatile organic compound contaminate down via a photo-catalytic oxidation process.

The air purification system 10 is designed to help sterilize the air and reduce indoor odors and volatile organic compound contamination from indoor air. By using an adsorption media, the air purification system 10 captures volatile organic compounds, as shown in FIG. 4, and then reduces the captured constituents through an innovative photo-catalytic oxidation process that breaks down the captured volatile organic compounds into elemental carbon dioxide and water vapor. The adsorption media may be, but is not limited to being a highly adsorptive activated carbon. In turn, the absorption media 12 may be continuously regenerated for on-going air treatment, as shown in FIG. 5. Additionally, the air purification system 10 may contain a UV light that irradiates the moving air stream helping to reduce airborne bacteria, viruses and allergens and organic odors, plus can eliminate any build up of mold from within air ducts 12 or air handlers 24.

The air purification system 10 may be formed from a housing 20 having one or more adsorptive photo-catalytic oxidation devices 12. The housing 20 may be formed from a generally rectangular box containing the adsorptive photo-catalytic oxidation device 12. The housing 20 may be formed from resilient materials such as, but not limited to, metals and plastics. A deflector 26 may extend from the housing 20 along the ultraviolet light source 18 to deflect air through the adsorptive photo-catalytic oxidation device 12 and to deflect ultraviolet radiation emitted from the ultraviolet light source 18. The deflector 26 may have any appropriate configuration. In at least one embodiment, as shown in FIG. 1, the deflector 26 may be a three sided device generally forming a U-shaped device. The deflector 26 may be formed from resilient materials such as, but not limited to, metals and plastics.

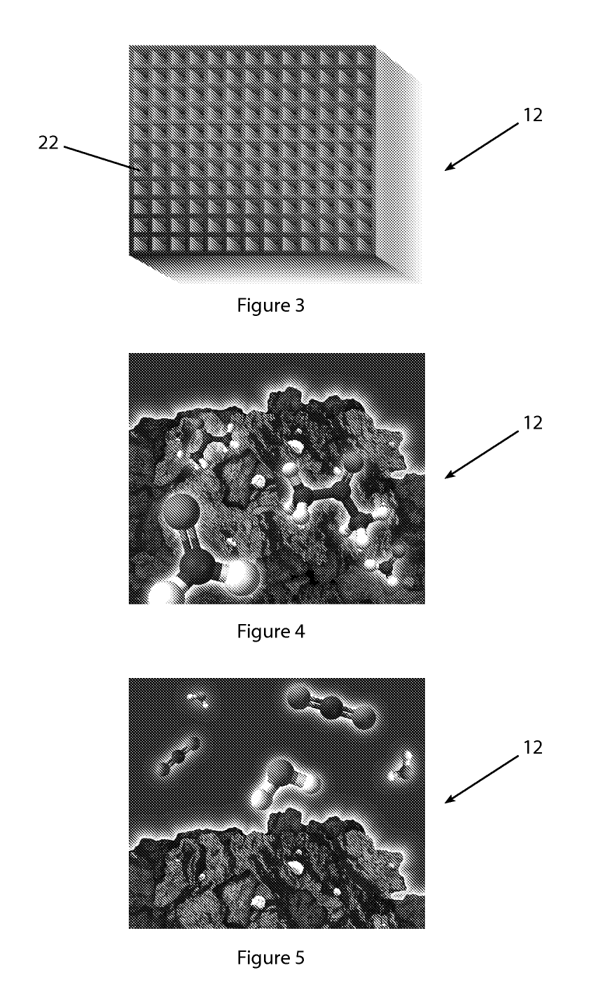

The adsorptive photo-catalytic oxidation device 12 may be formed from an adsorption media, as shown in FIG. 3, which may be a highly adsorptive activated carbon honeycomb monolithic media. In at least one embodiment, the adsorption media may be a carbon monolithic material. The adsorptive photo-catalytic oxidation device 12 may also include a coating 22 of a regenerative photo-catalyst on the adsorptive photo-catalytic oxidation device 12. The coating 22 of the regenerative photo-catalyst may be an ultraviolet reactive titanium dioxide based semi-conductor photo-catalyst or other form of precious metal semiconductor photo-catalyst material.

As shown in FIG. 1, the ultraviolet light source 18 may extend from the housing 20. The ultraviolet light source 18 may be positioned to expose the adsorptive photo-catalytic oxidation device 12 to ultraviolet light emitted by the ultraviolet light source 18 to breakdown captured volatile organic compounds into elemental carbon dioxide and water vapor and to irradiate air moving past the ultraviolet light 18 to reduce contaminants

The air purification system 10 may be used to clean air passing through an air handling system 16, as shown in FIG. 2. The housing 20 and ultraviolet light source 18 may be positioned in the air duct 14. In at least one embodiment, the ultraviolet light source 18 may be, but is not limited to, a mercury vapor style of light source, light emitting diodes (LED), xenon bulbs. The ultraviolet light source 18 may produce light in the UV-C germicidal spectrums such as 254 nM. This spectrum is effective in sterilizing microbial contaminates. The ultraviolet light source 18 may be positioned in an air duct 14 to provide the interior space of the central air handling system 16 with a way of controlling surface microbial contamination within the interior components of the unit. The ultraviolet light source 16 may produce light in the UV-C spectrum for the purpose of sterilization of microbial contamination.

In use, the air purification system 10 may be installed in the air duct 14 of one or more central air handling systems 16. As odors and chemical contaminates circulate through the air handling system 16, the air purification system 10 may utilize a highly adsorptive activated carbon monolithic media 12 that captures these contaminates removing them from the air stream, much like a sponge absorbs water.

Activated carbon adsorption is an effective method for removing gaseous contaminates. Although carbon is an extremely effective way of adsorbing airborne contaminates, it has a finite capacity to adsorb these contaminates. To overcome this limitation, the activated carbon monolithic media of the air purification system 10 has been coated with a regenerative photo-catalyst. This UV reactive titanium dioxide (TiO₂) based semi-conductor photo-catalyst, when exposed to ultraviolet light, becomes highly reactive and attacks the chemical bonds of volatile organic compounds and bio-aerosol pollutants, thereby reducing these gaseous airborne chemicals and biological contaminants to carbon dioxide (CO₂), and water vapor (H₂O). Other forms of precious metal semiconductor photo-catalyst material may be used as a catalyst. This process is referred to as photo-catalytic oxidation and is highly effective at breaking down complex volatile organic compounds. The air purification system 10 uses the absorption capabilities of carbon to absorb airborne volatile organic compounds and the catalytic oxidation ability of UV photo-catalytic oxidation technology to regenerate the carbon.

During the off cycles of the central air handling system 16, the self regenerating photo-catalytic process of the air purification system 10, breaks down the captured contaminates and frees up the activated carbon honeycomb monolithic cell to be able to capture additional airborne volatile organic compounds and odors. In addition to the ability of the air purification system 10 to absorb airborne volatile organic compounds, the ultraviolet light source 18 plays an important role in disinfecting and deodorizing the indoor air of any bacteria, viruses and allergens, reducing indoor air related allergies and illness. In addition, the ultraviolet light source 18 helps to maintain the cleanliness of the air handling system by shinning direct onto the ductwork, cooling coils, heat strips and blowers that are prone to have mold growth. During use, the ultraviolet light source 18 may irradiate ultraviolet light continuously or at intervals. The ultraviolet light may prevent growth and kill existing microbial contamination.

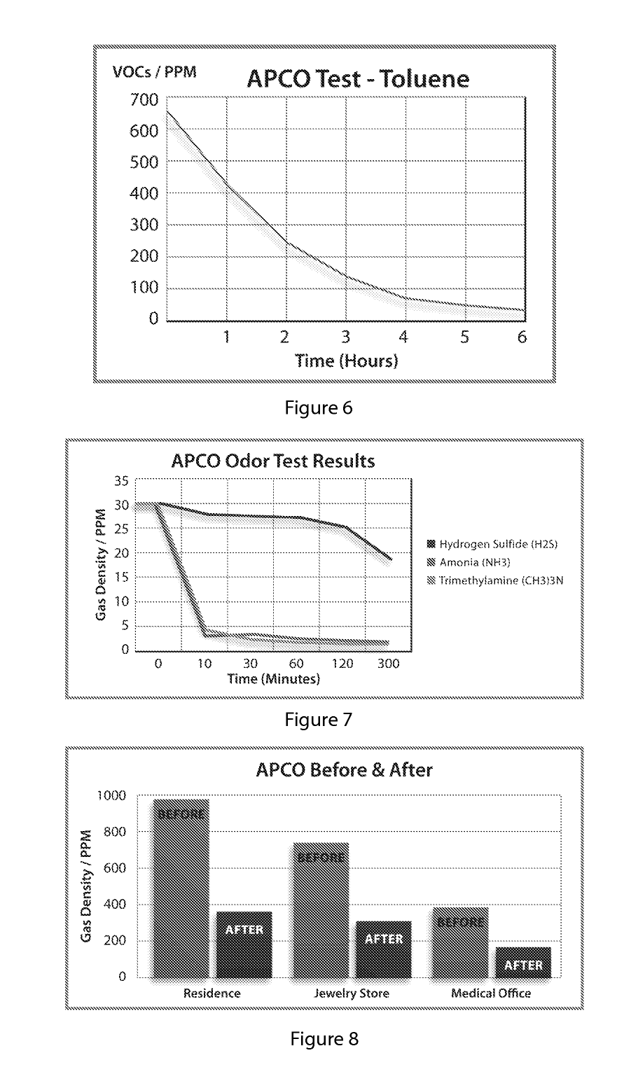

As shown in FIG. 6, the air purification system 10 may remove volatile organic compounds from air. In particular, air containing volatile organic compounds in amounts approaching 650 parts per million (ppm) may be reduced in about four hours to about 75 ppm. Further, the air purification system 10 may remove volatile organic compounds from amounts approaching 650 parts per million (ppm) in about six hours to about 35 ppm. The UV light source in the test was a 254 NM germicidal UV-C spectrum quartz hot filament. The photo-catalytic oxidation device was a monolithic absorptive cell with absorption media and TiO2 photo-catalyst. There were 16 cells per inch, 250 square meters/gm, a bulk density of 1.44 gm/cm2, a pressure drop of less than 0.005 in w.c. at 400 fpm, volatile organic compound activity of between 40% and 60% absorption per pass, and a maximum operating temperature of 400 degrees Fahrenheit.

FIG. 7 is a graph of the results of removing odor from air with the air purification system 10. Within about 10 minutes of passing air through the air purification system 10, the concentrations of ammonia and trimethylamine was reduced from about 30 ppm to about 3 ppm and about 4.5 ppm, respectively. Hydrogen Suflide was removed from air from a starting concentration of about 30 ppm to about 18 ppm over about 300 minutes.

FIG. 8 shows a graph of the results of the air purification system 10 of removing volatile organic compounds from air. In particular, the air purification system 10 may reduce volatile organic compounds in residences, jewelry stores, and medical offices from between about 57 percent and about 62 percent.

The foregoing is provided for purposes of illustrating, explaining, and describing embodiments of this invention. Modifications and adaptations to these embodiments will be apparent to those skilled in the art and may be made without departing from the scope or spirit of this invention.

I claim:

1. An air purification system, comprising:
a housing having at least one adsorptive photo-catalytic oxidation device; and
an ultraviolet light source positioned to expose the adsorptive photo-catalytic oxidation device to ultraviolet light emitted by the ultraviolet light source to breakdown captured volatile organic compounds into elemental carbon dioxide and water vapor and to irradiate air moving past the ultraviolet light and local surfaces to reduce contaminants

2. The air purification system of claim 1, wherein the at least one adsorptive photo-catalytic oxidation device is formed from an adsorption media.

3. The air purification system of claim 2, wherein the adsorption media is an activated carbon honeycomb monolithic material.

4. The air purification system of claim 1, further comprising a coating of a regenerative photo-catalyst on the at least one adsorptive photo-catalytic oxidation device.

5. The air purification system of claim 4, wherein the coating of a regenerative photo-catalyst is a ultraviolet reactive titanium dioxide based semi-conductor photo-catalyst.

6. The air purification system of claim 1, further comprising a central air handling system and an air duct extending therefrom, wherein the housing and ultraviolet light source are positioned in the air duct.

7. The air purification system of claim 1, wherein the housing is formed from a generally rectangular box containing the at least one adsorptive photo-catalytic oxidation device and wherein the ultraviolet light source extends from the housing.

8. The air purification system of claim 7, further comprising a deflector extending from the housing along the ultraviolet light source to deflect ultraviolet radiation emitted from the ultraviolet light source.

9. An air purification system, comprising:
a housing having at least one adsorptive photo-catalytic oxidation device with at least one outer surface exposed;
an ultraviolet light source positioned to expose the adsorptive photo-catalytic oxidation device to ultraviolet light emitted by the ultraviolet light source to breakdown captured volatile organic compounds into elemental carbon dioxide and water vapor and to irradiate air moving past the ultraviolet light and surfaces to reduce contaminants; and
a coating of a regenerative photo-catalyst on the at least one adsorptive photo-catalytic oxidation device.

10. The air purification system of claim 9, wherein the at least one adsorptive photo-catalytic oxidation device is formed from an adsorption media.

11. The air purification system of claim 10, wherein the adsorption media is an activated carbon honeycomb monolithic material.

12. The air purification system of claim 9, wherein the coating of a regenerative photo-catalyst is a ultraviolet reactive titanium dioxide based semi-conductor photo-catalyst.

13. The air purification system of claim 9, further comprising a central air handling system and an air duct extending therefrom, wherein the housing and ultraviolet light source are positioned in the air duct.

14. The air purification system of claim 9, wherein the housing is formed from a generally rectangular box containing the at least one adsorptive photo-catalytic oxidation device and wherein the ultraviolet light source extends from the housing.

15. The air purification system of claim 14, further comprising a deflector extending from the housing along the ultraviolet light source to deflect ultraviolet radiation emitted from the ultraviolet light source.

16. An air purification system, comprising:
a housing having at least one adsorptive photo-catalytic oxidation device with at least one outer surface exposed;
an ultraviolet light source positioned to expose the adsorptive photo-catalytic oxidation device to ultraviolet light emitted by the ultraviolet light source to breakdown captured volatile organic compounds into elemental carbon dioxide and water vapor and to irradiate air moving past the ultraviolet light and surfaces to reduce contaminants;
a coating of a regenerative photo-catalyst on the at least one adsorptive photo-catalytic oxidation device;
wherein the at least one adsorptive photo-catalytic oxidation device is formed from an adsorption media; and
wherein the coating of a regenerative photo-catalyst is a ultraviolet reactive titanium dioxide based semi-conductor photo-catalyst.

17. The air purification system of claim 16, wherein the adsorption media is an activated carbon honeycomb monolithic material.

18. The air purification system of claim 16, further comprising a central air handling system and an air duct extending therefrom, wherein the housing and ultraviolet light source are positioned in the air duct.

19. The air purification system of claim 16, wherein the housing is formed from a generally rectangular box containing the at least one adsorptive photo-catalytic oxidation device and wherein the ultraviolet light source extends from the housing.

20. The air purification system of claim 19, further comprising a deflector extending from the housing along the ultraviolet light source to deflect ultraviolet radiation emitted from the ultraviolet light source.
